(19) United States
(12) Patent Application Publication
(10) Pub. No.: US 2023/0335218 A1
(43) Pub. Date: Oct. 19, 2023

(54) SELF-DESIGNED SINGLE-NUCLEOTIDE POLYMORPHISM CHIP AND METHOD OF COMPUTING POLYGENICRISK SCORE FOR GIVEN POPULATIONS USING SELF-DESIGNED SINGLE-NUCLEOTIDE POLYMORPHISM CHIP

(71) Applicant: GENESTORY Joint Stock Company, Hanoi City (VN)

(72) Inventors: Tham Hong Hoang, Hanoi City (VN); Giang Minh Vu, Hanoi City (VN); Dat Thanh Nguyen, Hanoi City (VN); Trang Thi Ha Tran, Hanoi City (VN); Vinh Chi Duong, Hanoi City (VN); Nam Sy Vo, Hanoi City (VN)

(73) Assignee: GENESTORY Joint Stock Company, Hanoi City (VN)

(21) Appl. No.: 18/213,157

(22) Filed: Jun. 22, 2023

(30) Foreign Application Priority Data
May 10, 2023 (VN) 1-2023-03056

(51) Int. Cl. G16B 20/20; G16B 40/20

(52) U.S. Cl. CPC G16B 20/20; G16B 40/20

(57) ABSTRACT

The present invention relates to a self-designed single-nucleotide polymorphism chip and a method of computing polygenic risk score (PRS) for a given population using the self-designed single-nucleotide polymorphism chip. The self-designed single-nucleotide polymorphism chip using LmTag algorithm comprises the following modules: a pairwise imputation score computation module; a functional score computation module; and a tag SNP selection module. The method of computing polygenic risk score for a given population using the self-designed single nucleotide polymorphism chip comprises two computation flows: a first flow computing PRS based on a disease-/trait-related gene database collected from open sources and provided by parties; a second flow computing PRS based on test samples; wherein the self-designed SNP chip used in the VCF file generation stage of both flows; the VCF files is to be subjected to imputation, using a given population genomic dataset as a reference, and harmonized using a data harmonization process; and data generated from these two computation flows is to be harmonized and input into a machine learning model to form a single computation process to generate the polygenic risk score for a group of new test samples.

### CROSS-REFERENCES TO RELATED APPLICATIONS

This application claims priority from Vietnam Patent Application No. 1-2023-03056, filed May 10, 2023, the content of which is incorporated by reference herein in its entirety.

### FIELD OF INVENTION

The present invention relates to the field of bioinformatics, and particularly, to a self-designed single-nucleotide polymorphism chip and a method of computing polygenic risk score for a given population using the self-designed single-nucleotide polymorphism chip.

### BACKGROUND OF INVENTION

Genetic diseases are genetic disorders including syndromes, diseases, or malformations passed on by parents to their children or due to mutations in embryonic cells (during pregnancy). Genetic diseases occur due to genetic mutations or the structure and number of chromosomes. Abnormalities in the chromosomal structures, such as gene loss or excess, are the cause of the diseases; or random errors in one or more genes also cause genetic diseases. Genetic diseases are regarded by the medical community as intractable diseases worldwide.

The advancement in genetics has made it possible to generate polygenic predictions for complex traits in human, including the risks of numerous complex diseases. In genetics, the polygenic risk score (PRS), also referred to as the polygenic score (PGS), the polygenic index (PGI), etc., is intended to give an estimate of a person's likelihood of developing a particular disease, based solely on genetic factors without considering environmental factors. PRS is a highly focused and developing research area: hundreds of papers are published each year on topics such as algorithms for genomic prediction, construction of prediction tools, PRS applications, etc. In 2018, the American Heart Association (AHA) recognized PRS as one of the major research breakthroughs in the field of heart disease and stroke. Besides, PRS also contributes a lot to the research on obesity, diabetes, breast cancer, prostate cancer, Alzheimer's Disease and mental illnesses, etc. In addition, PRS has also demonstrated its effectiveness in diagnosing diseases and tailoring treatments to individual patients.

PRS is usually generated from genome-wide association studies (GWAS) data. To increase the efficiency in computing PRS, the researchers used a single-nucleotide polymorphism (SNP) chip to decode gene mutation points associated with complex diseases. Besides, SNP chip is also widely used because of the high accuracy of the technology.

However, because of the limited number of SNPs in the chip, tools for GWAS research often require a imputation step to increase the number of variants in test samples by predicting the genotypes of the variants that are not directly present in the SNP chip. Besides, the need to use international SNP chips poses the problem of cost and effectiveness on a given population's data.

In addition, at the present, PRS is less predictive in people of non-European ancestry since approximately 79% of the total GWAS participants are of European ancestry, although the European population accounts for only 16% of the global population (according to the GWAS Catalog). Therefore, it is necessary to develop an effective method of computing PRS for specific populations, especially for non-European populations.

### SUMMARY OF THE INVENTION

From the above fact, the present invention provides a self-designed SNP chip and a method of computing PRS for a given population using the self-designed SNP chip.

A self-designed SNP chip using an LmTag algorithm comprises the following modules:

a pairwise imputation score computation module that performs imputation as a linear model, harmonizes geneticinformation from the linkage disequilibrium squared correlation (LD r2), minor allele frequency (MAF), and physical distance between variants to give an imputation squared correlation value between an imputed genotype and a true genotype of the SNP (Imputation r2);

a functional score computation module that computes the functional scores for SNPs based on biological evidence from data sources: GWAS catalog, Clinvar and Combined Annotation Dependent Depletion (CADD) Score to assess the biological function for the SNP which did not have evidence;

a tag SNP selection module that selects a tag SNP based on two criteria:
having the largest CADD score among tag SNPs;
having the highest sum of the squared correlation between the imputed genotype and the true genotype of the SNP.

A method of computing PRS for a given specific population using the self-designed SNP chip, comprising steps that are divided into two flows:

a first flow computing PRS based on a disease-/trait-related gene database collected from open sources and provided by parties, comprising:

using data from the disease-/trait-related gene database collected from public source not necessary to mention “provided by parties”;

Calling genotype to generate a Variant Call Format (VCF) file;

performing imputation, normalization, and annotation on the VCF file to generate a post-processed VCF file;

converting the post-processed VCF file to a binary file (bfile), then computing PRS for all samples in the database;

a second flow computing PRS based on test samples, comprising:

using genetic data obtained from the test samples;

Calling genotype to generate a VCF file;

performing imputation, normalization, and annotation on the VCF file to generate a post-processed VCF file;

converting the post-processed VCF file to a bfile to compute PRS for the test sample;

wherein the self-designed SNP chip is used in the VCF file generation stage of both flows;

wherein the VCF file is needed to imputation, using a given population genomic dataset as a reference, and harmonized by using a data harmonization process;

wherein the new PRS-computed samples in the second flow are to be added to the existing sample set in the database;

data generated from these two computational flows is to be harmonized and input into a machine learning model to form a single computation process and generate PRS for a group of new test samples.

According to a particular embodiment of the present invention, the VCF file is computed using 1KVG (also known as VN1K—1000 Vietnamese Genome Project) and 1KGP (1000 Human Genome Project) datasets as reference, which contain whole genome sequencing (WGS) data for 1008 Vietnamese individuals (from the 1KVG project) and 2504 individuals (from the 1KGP project).

According to one aspect of the present invention, the self-designed SNP chip increases accuracy, prioritizes tag SNPs, has high utility for disease population, and reduces costs compared to international chips.

According to another aspect of the present invention, the LmTag algorithm used in the self-designed SNP chip not only uses statistical modeling to integrate information on LD r2,MAF, and physical distance between variants to increase the accuracy in tag SNP selection, but also identifies variants based on the pairwise imputation score and functional score to solve the problem regarding SNP selection.

According to another aspect of the present invention, the LmTag algorithm improves the imputation performance and prioritizes variant selection over existing methods for selecting tag SNP.

According to another aspect of the present invention, the method of imputation and annotation for the VCF file can significantly increase number of variants and overcome the limitations of the chip.

According to another aspect of the present invention, the harmonization of two computation flows of the method of computing PRS for a given population using the self-designed SNP chip reduces the computation time and cost for all existing samples and minimizes the processing time of new samples. In addition, the harmonization of the new samples into the existing sample set in the database increases the size of the existing sample dataset, thereby increasing the accuracy of the computation methods for the analysis process of the machine learning method.

According to another aspect of the present invention, the method of computing PRS for a given population using the self-designed SNP chip increases the accuracy of PRS computation for that given population, so it is highly appreciated in screening for genetic disease risks. Thus, it becomes useful for recommendation and treatment of patients, which contributes to an improvement in the patient's health.

According to another aspect of the present invention, after the method of computing PRS is performed, two evaluation parameters are obtained: a first parameter as the accuracy of imputation with the 1KVG and 1KGP datasets as reference; a second parameter as the accuracy of how well PRS can predict the disease/trait of the primary cohort in the first flow of the method, wherein the parameters for each gene table of the disease/trait can be estimated for better accuracy.

### BRIEF DESCRIPTION OF THE DRAWINGS

FIG. 1 shows a flowchart of a self-designed SNP chip;

FIG. 2 shows a flowchart of a method of computing PRS for a given population using the self-designed SNP chip according to an embodiment, using 1KVG and 1KGP datasets as reference;

FIG. 3 shows a flowchart of a data harmonization process;

FIG. 4 shows a flowchart of a machine learning model.

### DETAILED DESCRIPTION OF INVENTION

Below, the advantages, effects, nature, and aspects of the present invention can be better understood through a detailed description of particular embodiments based on the accompanying drawings. However, the present invention may be disclosed in various forms and should not be construed as being limited to any particular embodiment provided herein. Instead, these aspects are provided in full disclosure, and will completely convey the scope of the present invention to those skilled in the art.

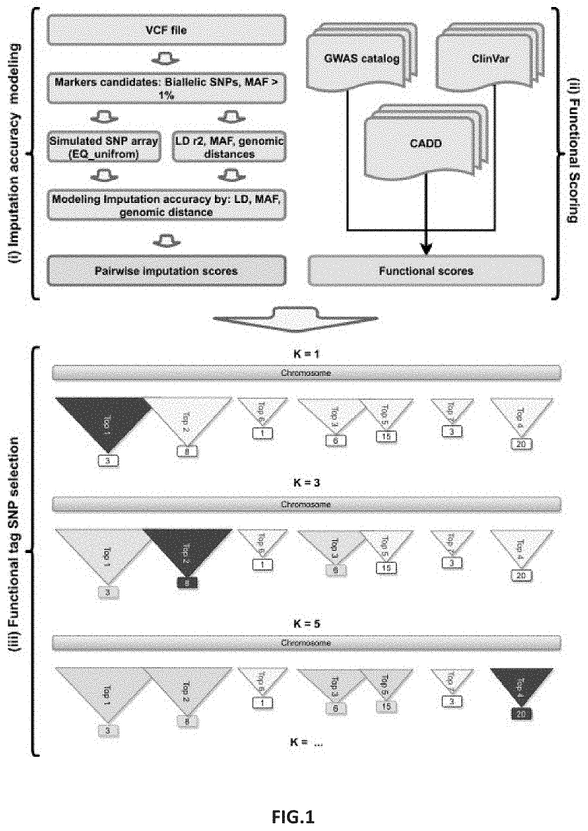

Referring to FIG. 1, a self-designed SNP chip using an LmTag algorithm comprises the following modules:

a pairwise imputation score computation module that performs imputation as a linear model, harmonizes information from LD r2, MAF, and physical distance between variants to give an imputation r2, as follows:

r=β₀+β₁·lᵢⱼ+β₂·mᵢ+β₃·mⱼ+β₄·dᵢⱼ (1)

wherein:

r is the imputation squared correlation between the imputed genotype and the true genotype of the sample (Imputation r2);

lᵢⱼ is the linkage disequilibrium squared correlation (LD r2) between the tag SNP and the tagged SNP (lᵢⱼ∈(0: 0.5]);

mᵢ is the minor allele frequency (MAF) of the tag SNP (mᵢ∈(0:0.5]);

mⱼ is the minor allele frequency (MAF) of the tagged SNP (mⱼ∈(0:0.5]);

dᵢⱼ is the physical distance between the tag SNP and the tagged SNP (dᵢⱼ∈N);

β₀, β₁, β₂, β₃, β₄ are the weights of the model (1);

a functional score computation module that computes the functional score for SNPs based on biological evidence from data sources: GWAS catalog, Clinvar and CADD Score to assess the biological function for the SNP which did not have evidence;

a tag SNP selection module that selects a tag SNP based on two criteria:

having the largest CADD score among tag SNPs;

having the highest sum of the imputation squared correlation between the imputed genotype and the true genotype of the SNP, particularly, the LmTag algorithm will independently compute the of each SNP in the pair of tag SNP and tagged SNP:

rᵢ=β₀+β₁·lᵢⱼ+β₂·mᵢ+β₃·mⱼ+β₄·dᵢⱼ (2)

wherein:

rᵢ is the imputation r² of the tag SNP;

rⱼ=β₀+β₁·lᵢⱼ+β₂·mⱼ+β₃·mᵢ+β₄·dᵢⱼ (3)

wherein:

rⱼ is the imputation r² of the tagged SNP;

then, the tag SNP with high CADD score is selected from a set A={SNP₁, SNP₂, SNP₃, . . . , SNPₙ}, for each SNP the values of imputation r² are summed up using the following formula:

Sᵢ=Σⱼ₌₁ⁿ [symbol] (4)

wherein:

lᵢⱼ≥c and i≠j;

Sᵢ is the sum of imputation r² values of the tag SNP;

[symbol] is the estimated value of imputation r² of the tagged SNP;

c is the constant set as the threshold;

then, a Beam Search algorithm is used to prioritize the selection of tag SNP with the highest CADD score among k SNPs with the highest total S in set A.

In this model, the imputation accuracy of the tagged SNP presumably depends on the tag SNP score. Accordingly, the relationship between the LD r2,the MAF of the tag SNP and the tagged SNP, and the physical distance between the variants are generated by simulation. One SNP array is generated and the accuracy of the tagged SNPs is computed, which retains the corresponding information of each SNP type and estimates the parameters of the linear model.

Next, the input variable is defined to include n SNPs, referred to as the following set: A={SNP₁, SNP₂, SNP₃, . . . , SNPₙ}. The SNPs are rearranged by position, then a subset comprising k SNPs is taken, called the tag SNP set: T={SNP₁, SNP₂, SNP₃, . . . , SNPₖ}. The remaining SNPs are labeled as the tagged SNP set: G={SNP₁, SNP₂, SNP₃, . . . , SNPₙ₋ₖ}. The computation accuracy of the tagged SNP belonging to set G is computed using a leave-one-out validation method. More particularly, imputation is performed using Minimac4 for each individual as long as the individual is not in the reference set. Tag SNPs belonging to the set T are defined as “identified genotype” and tagged SNPs belonging to set G are considered the “unknown genotype.” The accuracy of type computation for each tagged SNP belonging to the set G is expressed by an appropriate ratio, namely, the Pearson correlation coefficient (r2). To avoid confusion between LD r2, the correlation coefficient r2 between the dosage value of the genotype (0-2) and the reference dataset (0, 1, 2) is to be computed.

The linkage disequilibrium (LD) pair is to be computed by Plink v1.9 with a maximum intergenic distance of 1MB (Megabase) and a minimum LD r2 truncated by 0.2. MAF is to be computed and retrieved by bcftools. To simplify the model, each genotype of the tagged SNP is presumably computed based on the tag SNPs with the highest LD r2, thereby finding the tag SNP belonging to the best set T of each tagged SNP belonging to set G, then related information, including lᵢⱼ, MAF mᵢ, mⱼ LD pair and distance dᵢⱼ, is extracted.

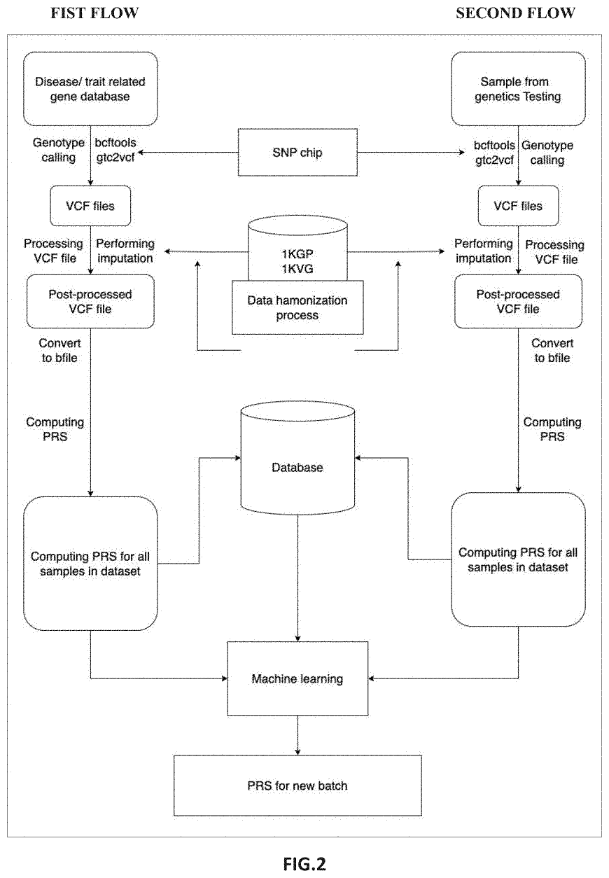

Referring to FIG. 2, a method of computing PRS for a given population using the self-designed SNP chip according to an embodiment using 1KVG and 1KGP datasets as reference, comprises steps that are divided into two flows:

a first flow computing PRS based a disease-/trait-related gene database collected from open sources and provided by parties, comprising:

using data from the disease-/trait-related gene database collected from open sources and provided by parties, wherein disease and trait related datasets is to be collected comprehensively, including descriptive data and genotypic data;

genotype calling by using bcftools and gtc2vcf to generate a VCF file;

performing imputation, normalization, and annotation of variant identifier (rsID) on the VCF file using an identified variant database (Single Nucleotide Polymorphism Database—dbSNP) to generate a post-processed VCF file;

converting the post-processed VCF file to a bfile, then computing PRS for all samples in the database;

a second flow computing PRS based on test samples, comprising:

using genetic data obtained from the test samples;

Calling genotypesgenotypic calling using bcftools and gtc2vcf to generate a VCF file;

performing imputation, normalization, and annotation on the VCF file by dbSNP to generate a post-processed VCF file;

converting the post-processed VCF file to a bfile to compute PRS for the test samples;

wherein the self-designed SNP chip is used in the VCF file generation stage of both flows;

wherein the VCF file is to be computed using 1KVG and 1KGP datasets as reference and harmonized using a data harmonization process;

wherein the new PRS-computed samples in the second flow is to be added to the existing sample set in the database;

data generated from these two computation flows is to be harmonized and input into a machine learning model to form a single computation process to generate PRS for a group of new test samples comprising (n+1) or more samples depending on the number of inputs.

In this method, imputation of the VCF file is a necessary step to increase the accuracy of the method, wherein the quality of the reference dataset plays very important role. A particular embodiment of the present invention is shown in FIG. 2, using a harmonized dataset from the 1KVG and 1KGP projects as reference, which contains the WGS data of 1008 Vietnamese individuals (from the 1KVG project, https://genome.vinbigdata.org) and 2504 individuals (from the 1KGP project) to ensure the quality of gene classification as well as complete coverage of specific subjects, and has been applied in Vietnam. The 1KVG project is known as the first large-scale human genome sequencing project in Vietnam with a sequencing range of greater than 30×. The harmonized data of these two projects increases the accuracy of imputation of the Vietnamese individual samples and the collected individual samples. Besides, missing rate of variants with >1% and MAF of variant with <0.1% are omitted in this harmonization

Besides, the data harmonization process also plays an essential role in the creation of post-processed VCF files. The harmonization process includes the following main steps: controlling the pre-imputation quality (Pre-imputation Quality Controls) of individuals and variants; performing imputation on unknown variants (Imputation); controlling the post-imputation quality (Post-imputation Quality Controls); harmonizing the datasets and then performing genotype re-imputation (Re-imputation).

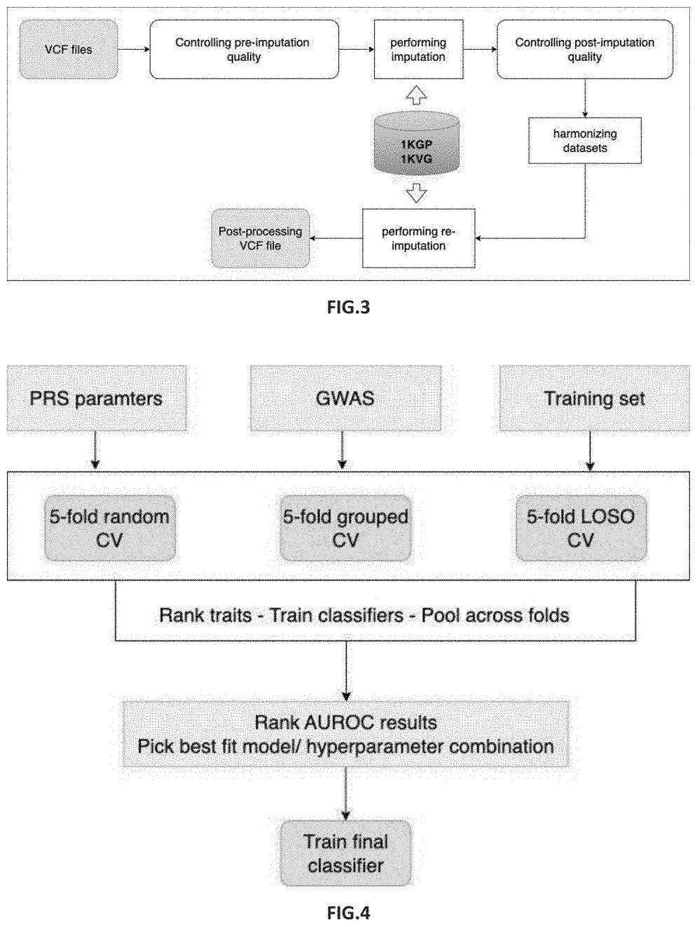

Referring to FIG. 3, the data harmonization process comprises the following particular steps:

controlling the pre-imputation quality of individuals and variants, comprising: filtering individuals by the heterozygosity, error-rate , missing rate, and Hardy-Weinberg p-value of the genotypic calling of different chips in order to reduce laboratory errors and improve the quality of genotypic data;

performing imputation for unknown variants, wherein the VCF file of each flow is to be imputed by Minimac4 with 1KVG and 1KGP datasets as reference;

controlling the post-imputation quality, comprising further removal of variants with low MAF and/or small Hardy-Weinberg p-value and/or low estimated squared correlation score between the imputed genotype and the true genotype of the sample (\hat{r}²);

harmonizing the datasets: all data after the post-imputation quality control step is to be merged and shared components of the datasets are to be removed (also known as the “Inner Join” method) to form a dataset without batch effects and low quality variants;

performing re-imputation similarly to the imputation step for unknown variants.

Referring to FIG. 4, a machine learning model is used to select, classify, and increase the number of reference samples for each trait from different data sources. Each new sample that meets the criteria, such as sample quality, race, age, gender, body mass index (BMI), etc., is to be classified and added to the reference sample set. Next, the refined machine learning model selects the parameters in the PRS computation, the GWAS set, and the reference sample datasets in the database. The data is to be selected, trained, and evaluated by random cross validation, grouped cross validation, and leave- one-out cross validation. The traits are to be ranked based on their contribution to the final classifier and summed up on the sections, and ROC curve (area under the receiver operating characteristic curve—AUROC) ranking result are used to find the best fitting and hyperparameter harmonization. After this training step, the final classifier of the training course is to be formed.

The particular embodiments of the invention provided herein are for illustrative purposes only. Those skilled in the art will understand well that various modifications can be made to the above embodiments without going beyond the principle and scope of the present invention. The scope of the present invention is defined by the accompanying claims.

We claim:

1. A self-designed single-nucleotide polymorphism (SNP) chip using an LmTag algorithm, comprising:
a pairwise imputation score computation module performing imputation as a linear model, harmonizing information from the linkage disequilibrium squared correlation (LD r²), minor allele frequency (MAF), and physical distance between variants to give an imputation squared correlation value between an imputed genotype and a true genotype of the SNP (Imputation r²);
a functional score computation module computing functional scores for SNPs based on biological evidence from data GWAS catalog, Clinvar, and Combined Annotation Dependent Depletion (CADD) Score to assess the biological function for SNP which did not have evidence;
a tag SNP selection module selecting a tag SNP based on:
having a largest CADD score among tag SNPs; and
having a highest sum of a squared correlation between an imputed genotype and an true genotype of the SNP.

2. A method of computing polygenic risk score (PRS) for a given population using the self-designed SNP chip according to claim 1, comprising steps that are divided into two flows:
a first flow computing PRS based on a disease-/trait-related gene database collected from open sources and provided by parties, comprising:
using data from the disease-/trait-related gene database collected from open sources and provided by parties;
genotypic calling to generate a Variant Call Format (VCF) file;
performing imputation, normalization, and annotation on the VCF file to generate a post-processed VCF file;
converting the post-processed VCF file to a binary file (bfile), then computing PRS for all samples in the database; and
a second flow computing PRS based on test samples, comprising:
using genetic data obtained from the test samples;
genotype calling to generate a VCF file;
performing imputation, normalization, and annotation on the VCF file to generate a post-processed VCF file;
converting the post-processed VCF file to a bfile to compute PRS for the test samples;
wherein the self-designed SNP chip is used in the VCF file generation stage of both flows;
wherein the VCF file is to be subjected to imputation using a given population genomic dataset as a reference and harmonized by using a data harmonization process;
wherein the new PRS-computed samples in the second flow are to be added to the existing sample set in the database;
data generated from these two computation flows is to be harmonized and input into a machine learning model to form a single computation process to generate PRS for a group of new test samples.

3. The method according to claim 2, wherein the VCF file is to be computed using 1KVG (also known as VN1K—1000 Vietnamese Genome Sequencing Project) and 1 KGP (1000 Human Genome Project) datasets as a reference.

4. The method according to claim 3, wherein the data harmonization process comprises the following main steps:
controlling the pre-imputation quality (Pre-imputation Quality Controls) of individuals and variants;
performing imputation for unknown variants (Imputation);
controlling the post-imputation quality (Post-imputation Quality Controls);
harmonizing the datasets; and
performing imputation (Re-imputation).

5. The method according to claim 4, wherein the data harmonization process comprises the following steps:
controlling the pre-imputation quality of individuals and variants, comprising: filtering individuals by the heterozygosity, error-rate, missing rate, and Hardy-Weinberg p-value of the genotypic calling of various chip arrays in order to reduce laboratory errors and improve the quality of genotypic data;
performing imputation for unknown variants, wherein the VCF file of each flow is to be computed by Minimac4 with 1KVG and 1KGP datasets as reference;
controlling the post-imputation quality, comprising further removal of variants with low MAF and/or small Hardy-Weinberg p (p-value) and/or low estimated square correlation score between the imputed genotype and the true genotype of the sample (\hat{r}²);
harmonizing the datasets: all data after the post-imputation quality control is to be merged and shared components of the datasets are to be removed (also known as the “Inner Join” method) to form a dataset without batch effects and low quality variants;
performing re-imputation similarly to the imputation step for unknown variants.

6. The method according to one of claim 5, wherein the machine learning model performs the following:
selecting, classifying, and increasing the number of reference samples for each trait from different data sources, wherein each new sample that meets the criteria, such as sample quality, race, age, sex, index body mass (BMI), etc., is to be classified and added to the reference sample set;
wherein the data is to be selected, trained, and evaluated by different cross-validation methods;
wherein the traits are to be ranked based on their contribution to the final classifier, summed up on the sections, and ROC curve (Area Under the Receiver Operating Characteristic Curve (AUROC) ranking results are used to find the best fitting and hyperparameter harmonization;
forming the final classifier of the training course.

7. The method according to claim 6, wherein the data of the machine learning model is selected from:
a PRS parameter;
a Genome-Wide Association Studies (GWAS) dataset;
a reference sample dataset in the database.

8. The method according to claim 7, wherein the cross-validation method of the machine learning model comprises:
performing random cross validation;
performing grouped cross validation; and
performing leave-one-out cross validation.
